(19) United States
(12) Patent Application Publication
(10) Pub. No.: US 2008/0208307 A1
(43) Pub. Date: Aug. 28, 2008

(54) Treatment of Vascular Bifurcations

(75) Inventors: Shmuel Ben-Muvhar, Emek Beit Shean (IL); Amir Miller, Ramat-Hasharon (IL)

Correspondence Address: DARBY & DARBY P.C., P.O. BOX 770, Church Street Station, New York, NY, 10008-0770, US

(73) Assignee: B-BALLOON LTD., Ariel (IL)

(21) Appl. No.: 10/595,648

(22) PCT Filed: Nov. 2, 2004
(86) PCT No.: PCT/IL04/01002

§ 371 Date: Jul. 7, 2006

Related U.S. Application Data
(60) Provisional application No. 60/517,213, filed on Nov. 3, 2003.
(60) Provisional application No. 60/607,064, filed on Sep. 3, 2004.

(51) Int. Cl. A61F 2/84; A61M 25/12; A61F 2/06; B29C 39/00

(52) U.S. Cl. 623/1.11; 604/103.07; 604/103.1; 604/103.05; 623/1.16; 264/299; 264/232; 264/239

(57) ABSTRACT

Apparatus for treatment of a vascular bifurcation, where a first blood vessel (24) meets a second blood vessel (26). The apparatus includes a balloon (40, 90) for deployment at the vascular bifurcation. The balloon includes a first part (42, 94), which has a first inflation characteristic and is adapted to be deployed in the first blood vessel, and a second part (44, 92), which has a second inflation characteristic, different from the first inflation characteristic, and is adapted to be deployed in the second blood vessel.

### CROSS REFERENCE TO RELATED APPLICATIONS

This application claims the benefit of U.S. Provisional Patent Application 60/517,213, filed Nov. 3, 2003, and U.S. Provisional Patent Application 60/607,064, filed Sep. 2, 2004. The disclosures of these related applications are incorporated herein by reference.

### FIELD OF THE INVENTION

The present invention relates generally to vascular catheterization, and specifically to intravascular balloons and stents.

### BACKGROUND OF THE INVENTION

Intravascular stents are used for various purposes, including opening occluded blood vessels. Typically, the stent is supplied in a narrow, contracted form, with a deflated balloon contained inside the stent. The stent and balloon are held at the distal end of a catheter. The physician inserts a guide wire into the blood vessel, and then slides the catheter over the wire to position the stent in the proper location. The balloon is then inflated, via a channel in the catheter, causing the stent to expand so as to be anchored in place and hold the vessel open. Once the stent has been expanded, the balloon is deflated and is withdrawn, along with the catheter, from the vessel.

It is sometimes necessary to insert a stent at the location of a bifurcation, where two blood vessels meet. In such cases, the stent must be inserted into the vessel that is to be expanded in such a way that the other vessel at the bifurcation is not blocked by the stent or damaged by the procedure. The physician performing the procedure must also take care not to dislodge plaques from either of the vessels at the bifurcation while performing the treatment.

The difficulty of treating vascular bifurcations is recognized in the alt, and a variety of solutions have been proposed. For example, U.S. Pat. No. 6,361,544, whose disclosure is incorporated herein by reference, describes a stent and catheter assembly for treating bifurcations. The stents described in the patent include stents for side-branch vessels, with an angulated portion that corresponds to the angle formed by the intersection of the side-branch vessel and the main vessel; main-vessel stents, which have an aperture that aligns with the opening to the side-branch vessel; and Y-shaped stents. Side-branch and main-vessel catheter assemblies are advanced over a pair of guide wires for delivering, appropriately orienting, and implanting the stents. A dual-balloon Y-shaped catheter is also described.

Bifurcated balloons for use in catheterization procedures are also described, for example, in U.S. Pat. Nos. 6,017,324, 6,210,380 and 6,123,718 and in U.S. Patent Application Publication 2003/0069561. The disclosures of these patents and patent application are incorporated herein by reference.

### SUMMARY OF THE INVENTION

Embodiments of the present invention provide novel balloons for treatment of vascular bifurcations, as well as methods for implanting stents in the area of a bifurcation using such balloons. (The term “bifurcation” as used herein refers to the area where two blood vessels meet, and includes the ostium.) These methods permit physicians to position stents with enhanced accuracy and ease. The balloons are also useful in reducing the likelihood that plaques will be released into the bloodstream during the procedure.

In some embodiments of the present invention, an intravascular balloon comprises two parts with different inflation characteristics. In other words, the two parts of the balloon are configured to respond differently to a given inflation pressure. Typically, when the balloon is inflated at a vascular bifurcation, one of the two parts of the balloon deploys into one vessel, while the other part deploys into the other vessel.

In some of these embodiments, the bifurcated balloon comprises a main, longitudinal part, with a radial protrusion at a predefined location and angle along the length of the main section. A stent is typically crimped over the balloon. The stent may have a side opening, such that when inflated, the radial protrusion of the balloon protrudes through the side opening of the stent. In one embodiment, the side opening is covered by radial struts, which open under pressure by the radial protrusion. The physician uses the protrusion to align the side opening of the stent with the side vessel at the bifurcation. Once the stent has been properly aligned in this manner, the balloon is fully inflated, causing the stent to expand and thus to be anchored in place, in optimal alignment with the side vessel. The balloon is then deflated and withdrawn.

Alternatively, the balloon may be used independently of a stent, for example, in balloon angioplasty procedures to open occluded blood vessels near bifurcations in the vessels. In this case, the radial protrusion of the balloon into the side vessel is still useful in aligning the balloon and preventing plaques at or near the bifurcation from breaking loose as one of the vessels is expanded. This added benefit of preventing plaque release may also be provided when the bifurcated balloon is used in expanding a stent, as described above.

In other embodiments, a balloon comprises a narrow inner part, for insertion into a side vessel at a bifurcation, and a collar, which surrounds one end of the narrow inner part of the balloon. The collar is configured to inflate to a larger diameter than the inner part. During treatment, the narrow part of the balloon is inserted into the side vessel so that the collar is positioned at the ostium, where the side vessel joins the main vessel. Inflation of the balloon causes the inner part to expand within the side vessel, while the collar, whose inflated diameter is larger than the side vessel, remains in the main vessel. In one embodiment, the inflated collar serves as a stop against the ostium, and thus aids the operating physician in positioning the stent properly at the ostium. In another embodiment, the balloon is used in implanting a novel stent in the side vessel, wherein one end of the stent protrudes from the side vessel into the main vessel and is expanded by the collar to a larger diameter than the rest of the stent in order to engage the ostium.

There is therefore provided, in accordance with an embodiment of the present invention, apparatus for treatment of a vascular bifurcation, where a first blood vessel meets a second blood vessel, the apparatus including a balloon for deployment at the vascular bifurcation, the balloon including:

a first part, which has a first inflation characteristic and is adapted to be deployed in the first blood vessel; and

a second part, which has a second inflation characteristic, different from the first inflation characteristic, and is adapted to be deployed in the second blood vessel.

In some embodiments, the second part is adapted to protrude radially from the first part when the balloon is inflated. Typically, the second part is adapted, upon partial inflation of the balloon, to extend into the second blood vessel so as to facilitate alignment of the balloon with the vascular bifurcation.

In one embodiment, the second part includes a fan-fold, which is adapted to unfold upon inflation of the balloon so that the second part extends into the second blood vessel. In another embodiment, while the balloon is deflated, at least a portion of the second part is contained inside the first part, and the second part is adapted to extend outward from the first part upon inflation of the balloon. In still another embodiment, the apparatus includes a retraction mechanism, which is coupled to the second part so as to retract the second part radially toward the first part upon deflation of the balloon.

In one aspect of the invention, the apparatus includes a radiopaque marker in at least a portion of the second pair, wherein the marker is configured so as to permit visualization of an alignment of the balloon relative to the bifurcation under angiographic imaging. In one embodiment, the radiopaque marker includes a ring encircling the second part.

In another embodiment, the apparatus includes a stent, which is fitted over the first part of the balloon and is adapted to be deployed within the first blood vessel by inflation of the balloon, wherein the stent has a radial opening to permit access between the first and second blood vessels, and wherein the second part of the balloon is adapted to protrude radially through the radial opening in the stent. The stent may be adapted to elute a therapeutic substance.

In one aspect of the invention, the inflation characteristic includes a degree of compliance, such that the first and second parts of the balloon have different, respective degrees of compliance. In one embodiment, the second part of the balloon includes a sleeve, which is secured over a portion of the first part of the balloon so as to constrain the compliance of the portion of the first part.

In some embodiments, the first and second blood vessels have characteristic first and second diameters, wherein the first diameter is greater than the second diameter, and wherein the first part of the balloon is adapted, upon inflation of the balloon while the second part is deployed in the second blood vessel, to assume an expanded diameter greater than the second diameter. In a disclosed embodiment, the first part of the balloon is configured as a collar around the second part of the balloon when the balloon is inflated. Typically, the first part of the balloon includes a toroid, which surrounds a portion of the second part of the balloon.

In some of these embodiments, the first part of the balloon is adapted, upon the inflation of the balloon, to engage an ostium. In one embodiment, the apparatus includes a stent, which is fitted over the second part of the balloon and is adapted to be deployed within the second blood vessel by the inflation of the balloon, the stent including a proximal end that is adapted to be expanded to a size greater than the second diameter, and the first part of the balloon is adapted to expand the proximal end of the stent so as to anchor the proximal end against the ostium. The proximal end of the stent may include a plurality of stunts, which are configured to be expanded to the size greater than the second diameter. In another embodiment, the first part of the balloon, when expanded, is adapted to serve as a stop against the ostium so as to aid in alignment of the stent within the second blood vessel.

Typically, at least one of the first and second parts of the balloon has a lumen passing therethrough to accommodate a guide wire used in the deployment of the balloon. In some embodiments, no more than one of the first and second parts of the balloon has the lumen passing therethrough. The first and second parts of the balloon share a common inflation port or may have separate, respective inflation ports.

There is also provided, in accordance with an embodiment of the present invention, a method for treatment of a vascular bifurcation, where a first blood vessel meets a second blood vessel, the method including:

providing a balloon including a first part, which has a first inflation characteristic, and a second part, which has a second inflation characteristic, different from the first inflation characteristic;

deploying the balloon at the vascular bifurcation, such that the first part is deployed in the first blood vessel and the second part is deployed in the second blood vessel; and

inflating the first and second parts of the balloon within the first and second blood vessels, respectively.

There is additionally provided, in accordance with an embodiment of the present invention, apparatus for treatment of a vascular bifurcation, where a first blood vessel meets a second blood vessel, the apparatus including:

a balloon for deployment at the vascular bifurcation, the balloon including a first part, which has a first inflation characteristic and is adapted to be deployed in the first blood vessel, and a second part, which has a second inflation characteristic, different from the first inflation characteristic, and is adapted to be deployed in the second blood vessel; and

a catheter, having a distal end to which the balloon is coupled, and which is adapted to pass through the first blood vessel so as to deploy the balloon at the bifurcation.

There is further provided, in accordance with an embodiment of the present invention, a method for manufacturing an intravascular balloon, including:

fabricating a first part of the balloon so as to have a first inflation characteristic; and

fabricating a second part of the balloon, coupled to the first part, so as to have a second inflation characteristic, which is different from the first inflation characteristic.

In one embodiment, fabricating the first and second parts of the balloon includes fabricating at least one of the first and second parts by injection molding using a bifurcated mold.

In another embodiment, fabricating the first and second parts of the balloon includes fabricating at least one of the first and second parts by blow molding using a bifurcated mold. The bifurcated mold may include a telescopic mold. Alternatively, fabricating the at least one of the first and second parts includes applying at least one of suction and an angled pin to direct material into the bifurcated mold.

In still another embodiment, fabricating the first and second parts of the balloon includes fabricating the first and second parts by dipping a bifurcated model in a liquid polymer.

Additionally or alternatively, fabricating the first and second parts of the balloon includes fabricating the first part of the balloon, and then applying a local treatment to an area of the first part in order to form the second part.

There is moreover provided, in accordance with an embodiment of the present invention, a vascular stent, including:

a distal section, which is adapted to be deployed and expanded within a blood vessel of a given diameter in a location adjacent to an ostium; and

a proximal section, which is adapted to be expanded against the ostium to a size greater than the given diameter so as to anchor the proximal section against the ostium.

In one embodiment, the distal section of the stent includes a first number of struts along a perimeter of the stent, and the proximal section of the stent includes a second number of the struts, greater than the first number. In another embodiment, the proximal section includes a plurality of struts, which are configured to bend outward so as to engage the ostium. At least one of the distal and proximal sections may be adapted to elute a therapeutic substance.

There is furthermore provided, in accordance with an embodiment of the present invention, apparatus for treatment of a vascular bifurcation, where a first blood vessel meets a second blood vessel, the apparatus including a balloon for deployment at the vascular bifurcation, the balloon including:

a first part, which is adapted to be deployed in the first blood vessel; and

a second part, which is adapted to protrude radially from the first part when the balloon is inflated so as to facilitate alignment of the balloon with the vascular bifurcation.

In some embodiments, the apparatus includes a stent, which is fitted over the first part of the balloon and is adapted to be deployed within the first blood vessel by inflation of the balloon, wherein the stent has a radial opening to permit access between the first and second blood vessels, and wherein the second part of the balloon is adapted to protrude radially through the radial opening in the stent. In one embodiment, the stent includes struts over the radial opening, and wherein the second part of the balloon is adapted to open the struts outward when the balloon is inflated.

There is also provided, in accordance with an embodiment of the present invention, apparatus for treatment of a vascular bifurcation, where a first blood vessel meets a second blood vessel, wherein the first and second blood vessels have characteristic first and second diameters, wherein the first diameter is greater than the second diameter, the apparatus including a balloon for deployment at the vascular bifurcation, the balloon including:

an inner part, which is adapted to be deployed in the second blood vessel; and

a collar around the inner part, which is adapted, upon inflation of the balloon while the second part is deployed in the second blood vessel, to assume an expanded diameter greater than the second diameter.

In some embodiments, the collar is adapted, upon the inflation of the balloon, to engage an ostium. In one embodiment, the apparatus includes a stent, which is fitted over the inner part of the balloon and is adapted to be deployed within the second blood vessel by the inflation of the balloon, the stent including a proximal end that is adapted to be expanded to a size greater than the second diameter, and the collar is adapted to expand the proximal end of the stent so as to anchor the proximal end against the ostium. In another embodiment, the collar, when expanded, is adapted to serve as a stop against the ostium so as to aid in alignment of the stent within the second blood vessel.

There is additionally provided, in accordance with an embodiment of the present invention, a method for treatment of a vascular bifurcation, where a first blood vessel meets a second blood vessel, the method including:

providing a balloon including a first part, which has a first inflation characteristic, and a second part, which is adapted to protrude radially from the first part when the balloon is inflated;

deploying the balloon in a vicinity of the vascular bifurcation, such that the first part is deployed in the first blood vessel;

partially inflating the balloon in the vicinity of the vascular bifurcation so that the second part protrudes radially away from the first part; aligning the second part of the partially-inflated balloon with the second blood vessel; and

fully inflating the balloon after aligning the second part.

There is further provided, in accordance with an embodiment of the present invention, a method for treatment of a vascular bifurcation, where a first blood vessel meets a second blood vessel, wherein the first and second blood vessels have characteristic first and second diameters, wherein the first diameter is greater than the second diameter, the method including:

providing a balloon including an inner part and a collar around the inner part;

deploying the balloon at the vascular bifurcation, such that the inner part is deployed in the first blood vessel and the collar is deployed in the second blood vessel; and

inflating the balloon so that the collar expands to an expanded diameter greater than the second diameter and engages an ostium.

The present invention will be more fully understood from the following detailed description of the embodiments thereof, taken together with the drawings in which:

### BRIEF DESCRIPTION OF THE DRAWINGS

FIGS. 1-3 are schematic side views of a stent and balloon used in implanting the stent at a bifurcation in a blood vessel, at successive stages in the process of implantation, in accordance with an embodiment of the present invention;

FIG. 4 is a schematic, pictorial view of a stent for implantation at a bifurcation, in accordance with another embodiment of the present invention;

FIGS. 5-7 are schematic pictorial illustrations of a balloon that is aligned with and inflated within a bifurcation in a blood vessel, at successive stages in the process of alignment and inflation, in accordance with another embodiment of the present invention;

FIG. 8 is a schematic, sectional view of a balloon with a radial protrusion, in accordance with an embodiment of the present invention;

FIG. 9 is a schematic, pictorial illustration showing insertion of a balloon into a bifurcation in a blood vessel, in accordance with an embodiment of the present invention;

FIGS. 10 and 11 are schematic, pictorial illustrations showing a balloon with a radial protrusion and a mechanism for retraction of the radial protrusion at successive stages in the process of insertion of the balloon into a bifurcation of a blood vessel, in accordance with an embodiment of the present invention;

FIG. 12 is a schematic, pictorial illustration of a stent, in accordance with an embodiment of the present invention;

FIG. 13 is a schematic, pictorial illustration of an intravascular balloon, in accordance with an embodiment of the present invention;

FIGS. 14 and 15 are schematic, pictorial illustrations showing successive stages in a process of implanting a stent in a side vessel at a bifurcation, in accordance with an embodiment of the present invention;

FIG. 16 is a schematic, exploded view showing parts of an intravascular balloon, in accordance with another embodiment of the present invention;

FIG. 17 is a schematic, pictorial illustration of a stent, in accordance with another embodiment of the present invention; and

FIGS. 18 and 19 are schematic, pictorial illustrations showing successive stages in a process of implanting a stent in a side vessel at a bifurcation, in accordance with another embodiment of the present invention.

### DETAILED DESCRIPTION OF EMBODIMENTS

FIG. 1 is a schematic side view of a stent 20, which is inserted into a blood vessel 24 at the location of a bifurcation in the vessel, in accordance with an embodiment of the present invention. The bifurcation in this example is the meeting point of vessel 24 with a side vessel 26. Vessel 24 is referred to hereinbelow as the “main vessel,” because it is the blood vessel through which stent 20 is inserted into the bifurcation. Typically, the main vessel has a larger diameter and carries a relatively larger volume of blood than does side vessel 26. In general, however, the principles of the present invention may be applied in treating both “main vessels” and “side vessels” in a bifurcation, regardless of the relative sizes of the vessels. In other words, the terms “main vessel” and “side vessel” are used in the present patent application and in the claims solely for convenience and clarity of explanation, and should not be construed as limiting the applicability of embodiments of the present invention to one sort of blood vessel or another.

Stent 20 is typically crimped on a balloon 32, which is inserted over a guide wire 22 into vessel 24 in order to treat plaques 30 obstructing the vessel in the area of the bifurcation. Initially, during insertion of the stent through vessel 24, balloon 32 remains deflated, and the stent has a narrow diameter, as shown in FIG. 1. The stent has a side opening 28 that must be aligned with side vessel 26 at the bifurcation in order to permit access to the side vessel, to enable blood flow in the side vessel after the stent has been expanded, and possibly additional treatment in the side vessel, as well. (The side opening of the stent may optionally be initially closed by a suitable structure, such as radial struts, as shown in FIG. 4.) The stent is typically constructed from a biocompatible metal or other rigid, expandable material, and may be configured to elute a therapeutic substance following implantation, using methods and formulations known in the art.

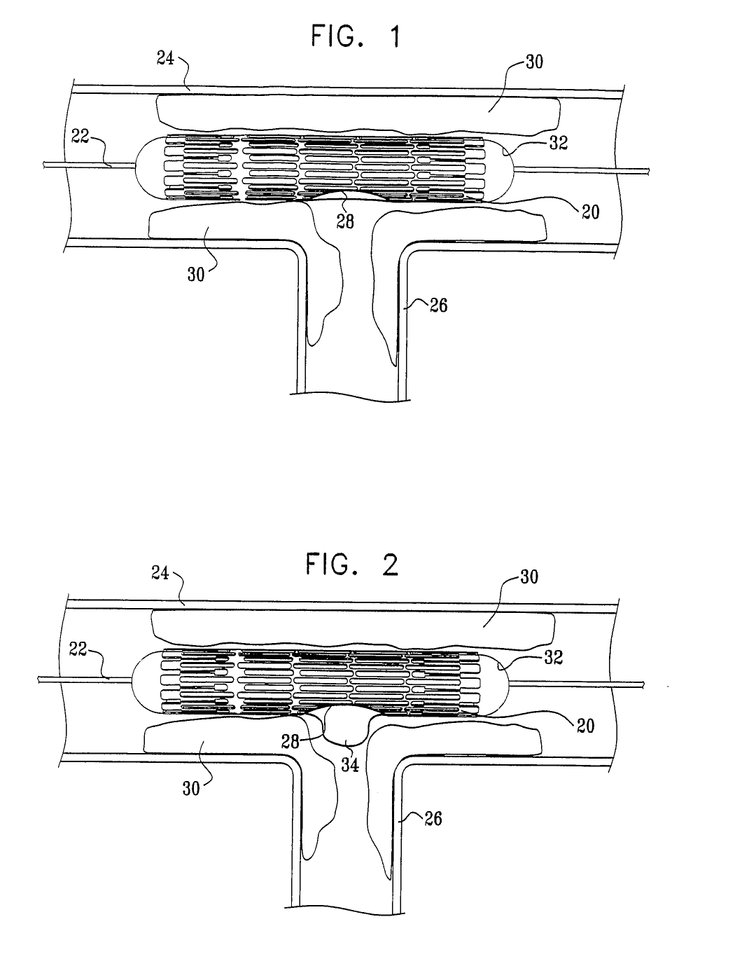

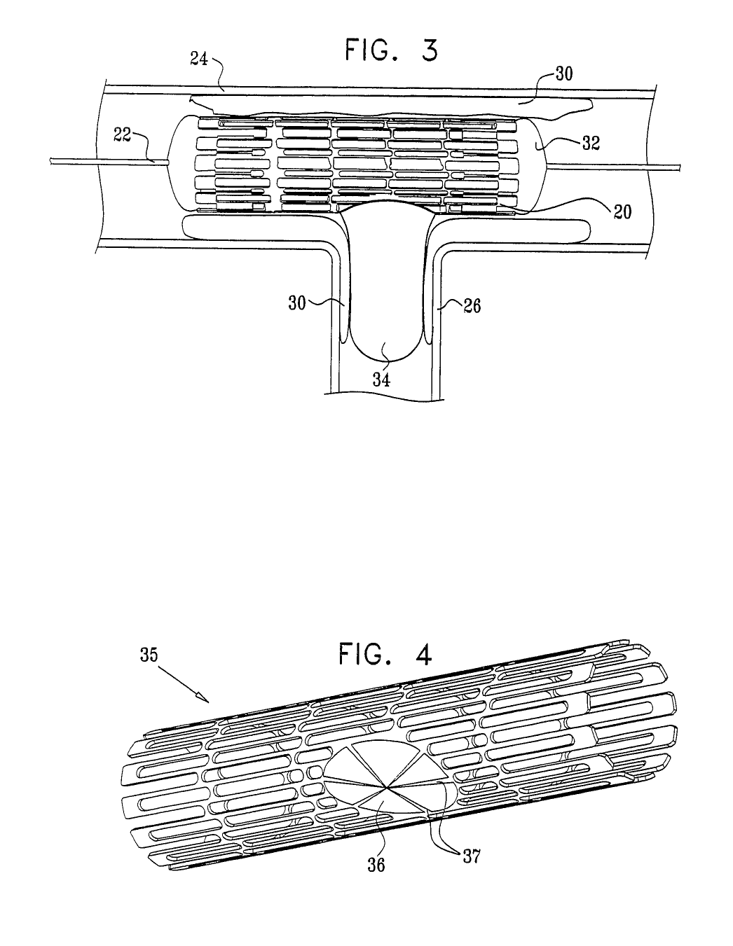

Reference is now made to FIGS. 2 and 3, which are schematic side views of stent 20 in blood vessel 24 in successive stages of implantation of the stent, in accordance with an embodiment of the present invention. In the stage shown in FIG. 2, balloon 32 inside stent 20 is partially inflated, causing a radial protrusion 34 of the balloon to protrude through the side opening of the stent. This radial protrusion may be made inherently more compliant than the main body of balloon 32 by appropriate treatment of the balloon at the time of manufacture. (Methods of manufacture that may be used for this purpose are described hereinbelow.) Alternatively, the radial protrusion may be more compliant simply because it is located inside opening 28 and thus is not constrained by the stent. Typically, in the stage shown in FIG. 2, the balloon is inflated to a low pressure, for example, about ¼ atm, via a fluid channel in the catheter (not shown) that is used to insert the stent. The low pressure is sufficient to cause the radial protrusion to expand through the side opening, but is not sufficient to cause the main body of the balloon (inside the stent) to make the stent itself expand.

The operating physician performs two steps in order to align side opening 28 of stent 20 with side vessel 26: rotation of the stent about its longitudinal axis, and longitudinal motion of the stent along the axis. Typically, the physician uses X-ray imaging or other radiographic imaging of the blood vessels and stent for assistance during these steps. Additionally or alternatively, protrusion 34 of the balloon may give the physician tactile feedback, indicating when the protrusion has entered the opening of the side vessel. In some embodiments, the balloon is partially inflated, as shown in FIG. 2, prior to the rotation step. In other embodiments, the balloon is partially inflated only after the stent has been turned to the proper orientation, and the protrusion of the balloon is thus used primarily for longitudinal alignment of the side opening of the stent with the side vessel.

In some embodiments, balloon 32 is inflated with a radiopaque fluid, such as saline solution mixed with a suitable contrast agent. The operating physician is then able to see the balloon—and in particular to see the location of radial protrusion 34 of the balloon—under X-ray imaging. Until side opening 28 of stent 20 is properly positioned adjacent to the entrance of side vessel 26 at the bifurcation, the radial protrusion of the balloon will be at least partly compressed by the walls of main vessel 24 or by plaques 30 within the vessel. When the side opening of the stent is properly aligned, however, the physician will see that the radial protrusion of the balloon has expanded outward into the side vessel, as shown in FIG. 2.

After stent 20 has been correctly positioned using the partially-inflated balloon 32, the balloon is inflated to full pressure, as shown in FIG. 3. For example, the pressure in the balloon may be increased at this point to about 1.5 to 2 atm, which is typically sufficient to expand the stent. Radial protrusion 34 of the balloon expands further under the increased pressure, and presses against the plaques in the area of the bifurcation. Expansion of the balloon protrusion has two desirable effects: (1) During expansion of the stent, the protrusion holds side opening 28 of the stent in precise alignment with side vessel 26. (2) The balloon helps to prevent collapse of the walls of side vessel 26 and to prevent plaques 30 from breaking loose from the vessel walls while the stent is being expanded. When the side vessel walls collapse or plaques do break loose, dangerous and even fatal consequences may result downstream. These latter anti-embolic effects of the bifurcated balloon are also useful when the balloon alone is used to expand a bifurcated vessel, even in the absence of a stent.

Once the stent has been fully expanded, balloon 32 is deflated and is then withdrawn from the vessel, leaving stent 20 in place. (Guide wire 22 is also withdrawn, of course.) Radial protrusion 34 of the balloon is made small enough so that upon deflation, it is drawn back through side opening 28 of the stent, without risk of being stuck in place. As noted above, the protrusion typically has a different inflation characteristic from the remainder of balloon 32 in order to facilitate the process of differential inflation described above. For example, protrusion 34 may be made of a flexible but relatively inelastic material, to prevent it from being overinflated when high pressure is applied to expand the stent.

FIG. 4 is a schematic, pictorial illustration of a stent 35, in accordance with another embodiment of the present invention. Stent 35 has a side opening 36, which is initially closed by radial struts 37. Once stent 35 is properly located in a vascular bifurcation, inflation of the radial protrusion of the balloon (not shown in this figure) causes struts 37 to open outward into the side vessel thus supporting the ostium. The balloon is subsequently deflated and withdrawn.

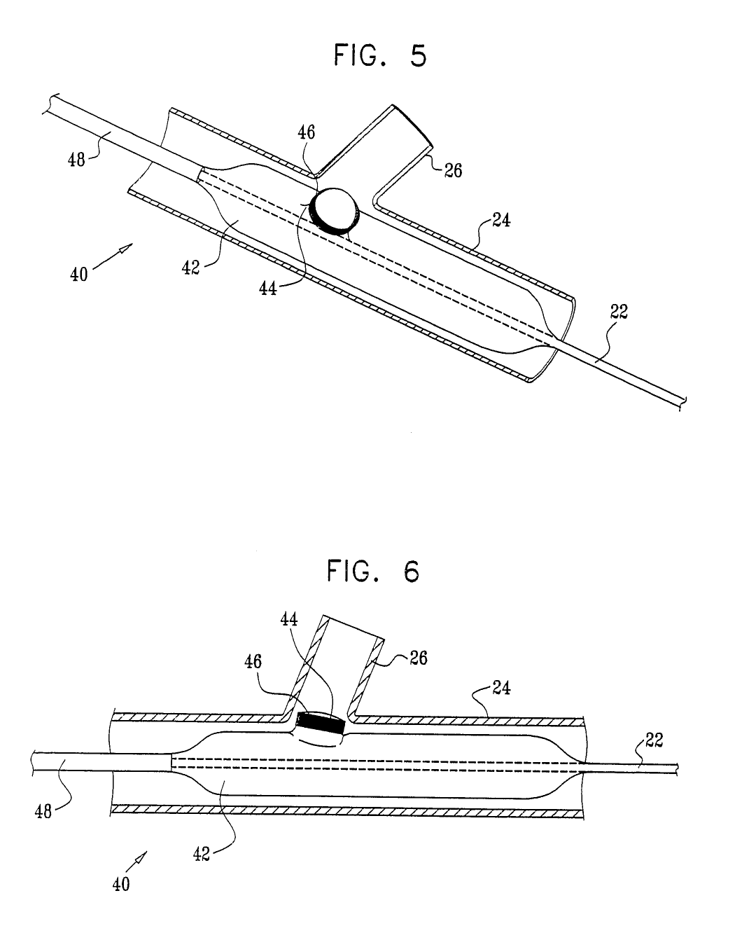

FIGS. 5 and 6 are schematic side views illustrating insertion and alignment of a balloon 40 at a bifurcation of vessels 24 and 26, in accordance with an embodiment of the present invention. Balloon 40 may be used in conjunction with a stent, as in the preceding embodiment, or on its own as shown in FIGS. 5 and 6. The balloon comprises a main body 42 and a radial protrusion 44, which is encircled by a radiopaque marker 46. The balloon in this case, too, is designed to be inserted into the area of the bifurcation by a catheter 48 over guide wire 22, without the assistance of a guide wire in side vessel 26. Alternatively, a guide wire may be inserted into side vessel 26 in addition to or instead of guide wire 22 in vessel 24, or the balloon may be inserted in the bifurcation without the use of a guide wire.

In one embodiment, marker 46 comprises a wire coil, which encircles protrusion 44. For example, the coil may comprise a superelastic shape-memory alloy, such as Nitinol, which is fabricated so that normally, in the absence of external forces, the coil has the compressed shape shown in FIGS. 5 and 6. Typically, the coil is embedded in the balloon material. Alternatively, the coil may be positioned either inside or outside protrusion 44. Protrusion 44 may have a fan-fold form, with multiple accordion-like folds. In order to align protrusion 44 with side vessel 26, the operating physician observes the area of the bifurcation using a suitable imaging system, such as an angiography system or other type of fluoroscope. The imaging system is aligned so that the image plane is parallel to the plane containing both of vessels 24 and 26 at the bifurcation, i.e., the plane of the page in FIGS. 5 and 6. In FIG. 5, protrusion 44 has not yet been aligned with side vessel 26, and marker 46 therefore appears in the angiographic image as an ellipse. The physician rotates catheter 48 until protrusion 44 is aligned with the opening of vessel 26, whereupon marker 46 appears as a straight line, as shown in FIG. 6.

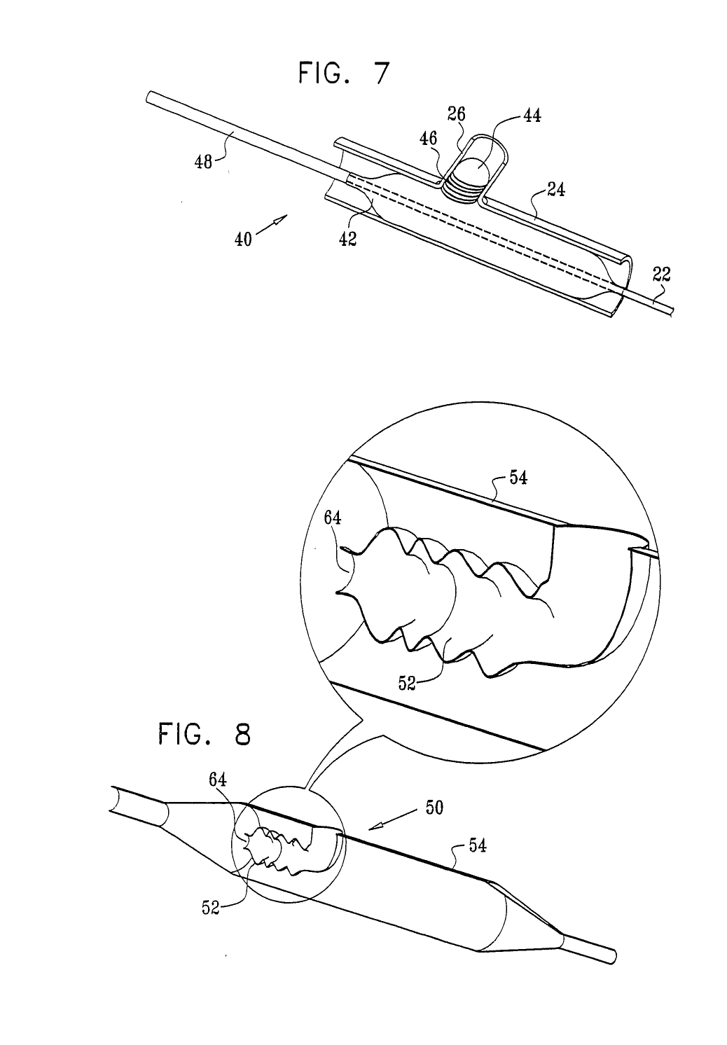

FIG. 7 is a schematic side view showing inflation of balloon 40 inside the bifurcation. The balloon is inflated, as in the other embodiments described herein, by injection of a suitable fluid through catheter 48. The inflation pressure causes protrusion 44 to unfold and thus expand into side vessel 26. As a result, marker 46 expands, so that the turns of the coil are separated from one another. After the procedure is completed, and the balloon is deflated, the coil contracts back to its original shape, pulling protrusion 44 in toward main body 42 of balloon 40, and thus facilitating its removal from the body.

Alternatively, other means may be used to mark protrusion 34 or 44 for the purposes of alignment with bifurcation 26. For example, instead of a wire coil, marker 46 may comprise one or more wire rings. Further alternatively, a radiopaque paint or dye may be embedded in the wall of the balloon in the area of the protrusion. In one embodiment, marker 46 comprises a ring of radiopaque paint around the base of protrusion 46, which will have a similar appearance under angiography to the wire coil described above. Further alternatively, protrusion 44 may comprise a central lumen to accommodate a guide wire, as shown in the next embodiment.

FIG. 8 is a schematic, pictorial illustration of a balloon 50, in accordance with another embodiment of the present invention. Balloon 50 comprises a main body 54 and a radial protrusion 52, as in the preceding embodiments. In this case, however, until balloon 50 is inflated inside the bifurcation, radial protrusion 52 is inverted and is thus contained inside main body 54. In this configuration, the radial protrusion is inverted, so that it appears as an indentation, rather than a protrusion. The tip of radial protrusion 52 has an opening 64 to permit an inner tube (not shown in this figure) containing a guide wire to pass through the radial protrusion into side vessel 26. Main body 54 of balloon 50 may contain an additional lumen (not shown) to accommodate another guide wire in the main vessel. As noted earlier, balloon 50 may be used to treat vascular bifurcations with or without an accompanying stent.

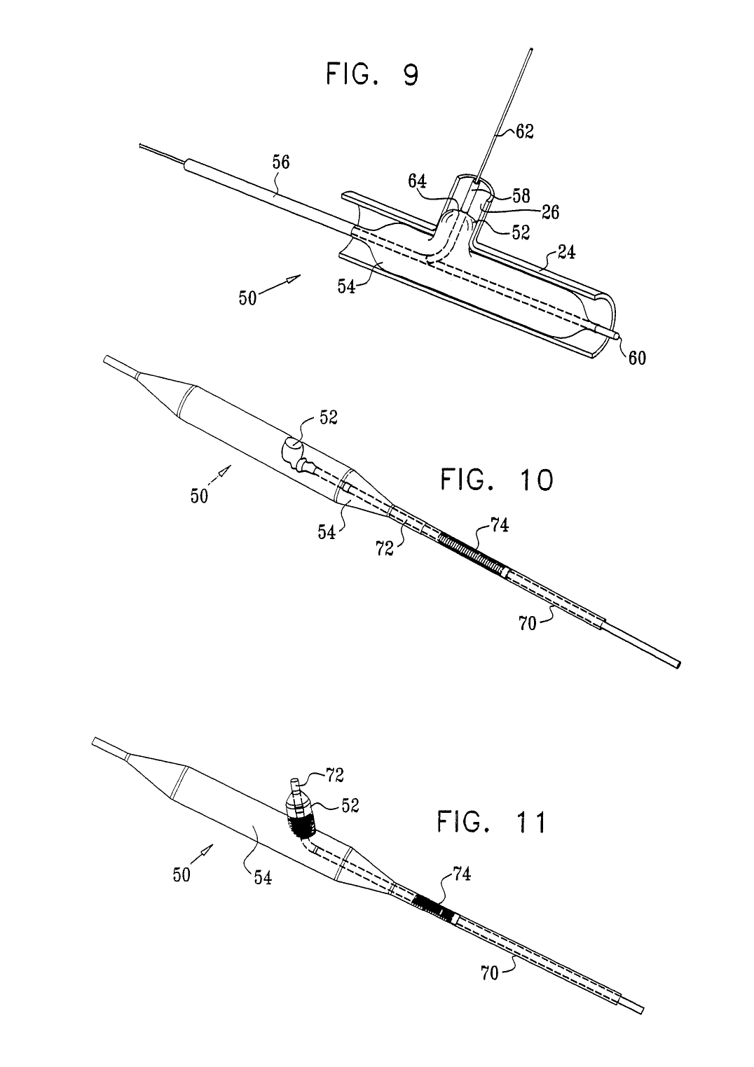

FIG. 9 is a schematic side view of the bifurcation of vessels 24 and 26, showing deployment of balloon 50 within the bifurcation, in accordance with an embodiment of the present invention. A catheter 56, which is used to deploy balloon 50, comprises an inner tube 58, which passes through opening 64 in the tip of protrusion 50. In the embodiment shown in FIG. 9, main body 54 of balloon 50 fits over a blind termination 60 at the distal end of the catheter. Alternatively, the tip of main body 54 may be open to accommodate a guide wire, as in the preceding embodiments. To insert balloon 50 in the bifurcation, a guide wire 62 is first inserted through vessel 24 into vessel 26, as shown in the figure. Catheter 56, with balloon 50 in its deflated state (as shown in FIG. 8), is then advanced over guide wire 62 into the area of the bifurcation, so that inner tube 58 passes into side vessel 26, while termination 60 remains in main vessel 24. Inflation of balloon 50 then causes protrusion 52 to evert out of its initial position inside main body 54, shown in FIG. 8, to the expanded configuration shown in FIG. 9.

Alternatively, a balloon with a non-everting protrusion, as shown in FIGS. 5 and 6, for example, may be inserted over a guide wire and inflated in the manner shown in FIG. 9.

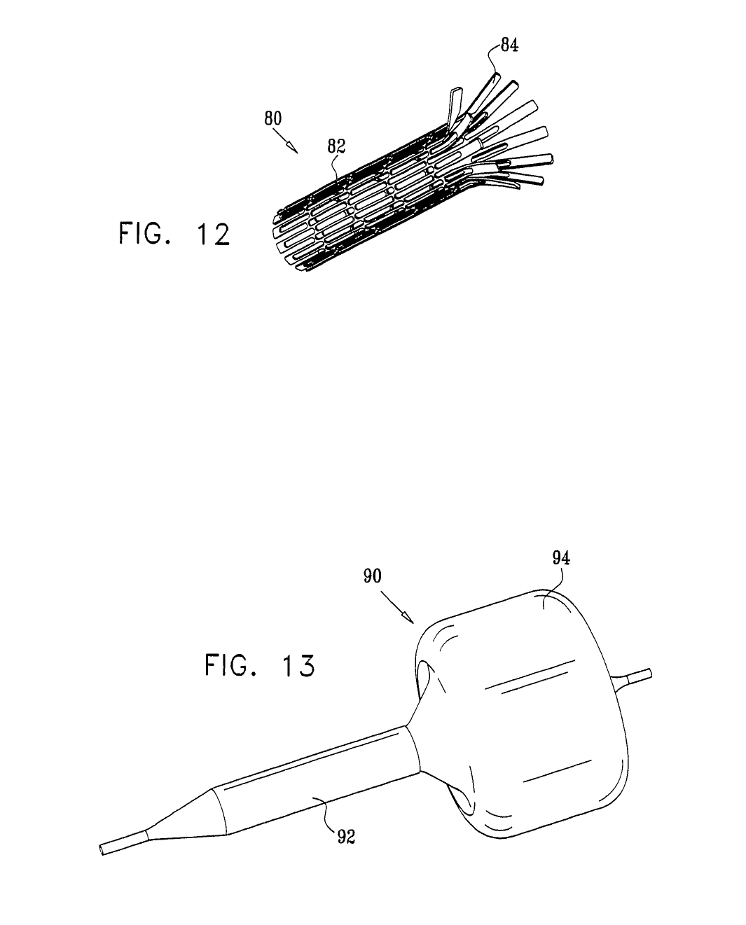

FIGS. 10 and 11 are schematic, pictorial illustrations showing a mechanism 70 that may be used to assist in retraction of radial protrusion 52 after use, in accordance with an embodiment of the present invention. FIG. 10 shows protrusion 52 and mechanism 70 in the retracted position, while FIG. 11 shows the protrusion mechanism in the expanded position. Although mechanism 70 is shown here in conjunction with balloon 50, similar mechanisms may be adapted for use with other types of radial protrusions that are described herein.

Mechanism 70 comprises an articulating retraction arm 72, which is attached at its distal end to the tip of protrusion 52 and is held at its proximal end by a spring. Initially, as shown in FIG. 10, during insertion of balloon 50 into the area of the bifurcation, spring 74 is relaxed, and arm 72 is thus retracted, holding protrusion 52 in its inverted configuration inside main body 54 (i.e., in the configuration shown in FIG. 8). Inflation of balloon 50 causes protrusion 52 to extend out of main body 54. Extension of protrusion 52 pulls am 72 outward along with it, in the distal direction, and compresses spring 74, as shown in FIG. 11. (Spring 74 is designed so that the tensile force it exerts is small enough to be overcome by the inflation pressure of protrusion 52.) When balloon 50 is finally deflated, at the end of the procedure, spring 74 pulls arm 72 back in the proximal direction, and thus pulls protrusion 52 back to its initial position inside main body 54, as shown in FIG. 10. In this position, the operating physician can withdraw balloon 50 from the patient's body without interference by protrusion 52.

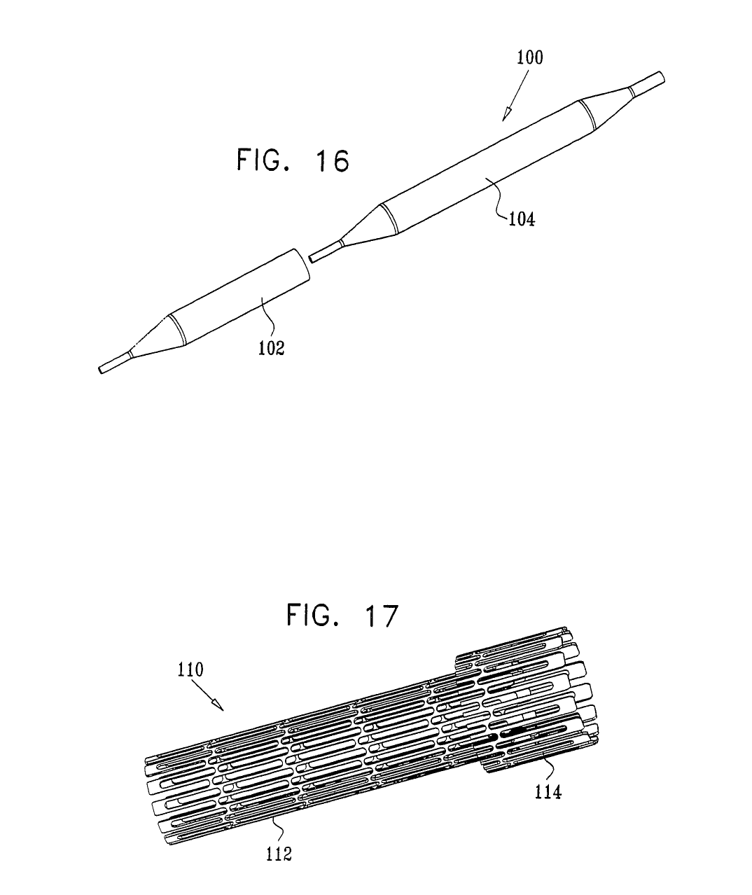

FIG. 12 is a schematic, pictorial illustration of a stent 80 for implantation in a side vessel at a bifurcation, in accordance with an embodiment of the present invention. Stent 80 is designed to engage the ostium, as described hereinbelow. As in other embodiments, stent 80 comprises a suitable biocompatible material, which may be capable of eluting a therapeutic substance following implantation. Stent 80 comprises a distal section 82 of conventional construction and a proximal end made up of struts 84, which are capable of deforming outward to fit the shape of the ostium, as shown in FIG. 15 below. Typically, the struts may bend outward by as much as 90°. During delivery of the stent through the vascular system, however, the entire stent, including struts 84, is maintained in a contracted configuration, with an approximately constant diameter over the entire length of the stent.

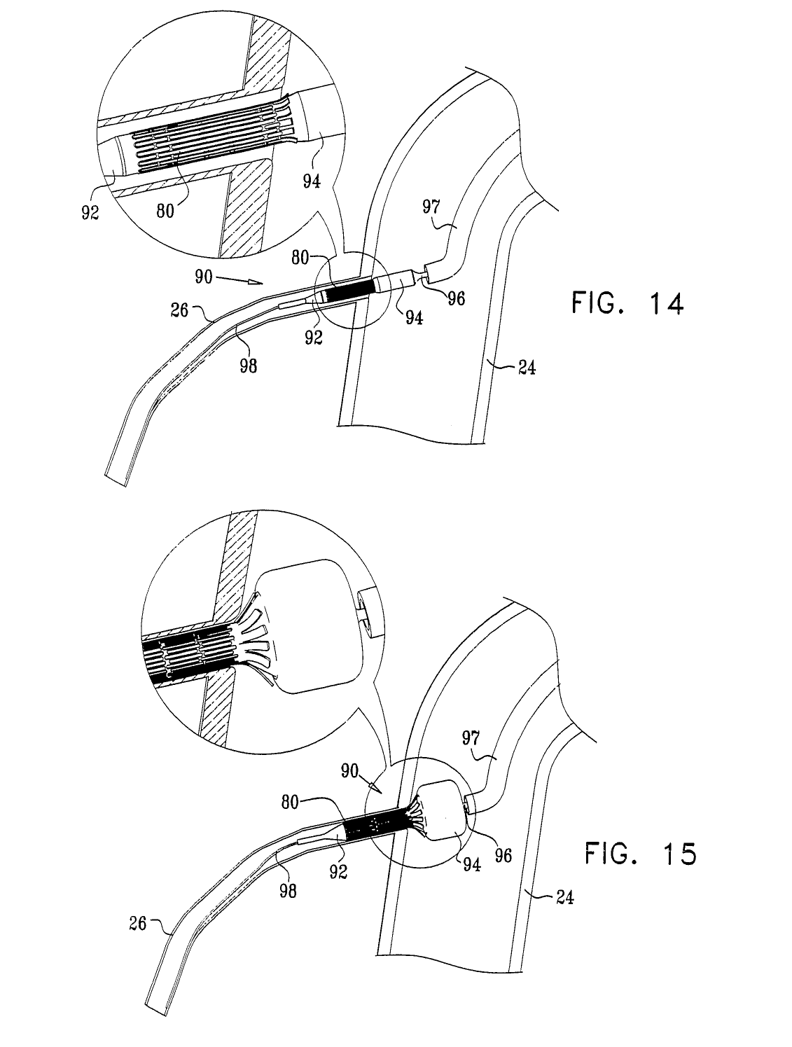

FIG. 13B is a schematic, pictorial illustration of a balloon 90, for use in intravascular treatment in the area of a bifurcation, in accordance with an embodiment of the present invention. The balloon is designed to engage the ostium in the bifurcation. Balloon 90 may be used, for example, in implanting stent 80, as described below. Alternatively, balloon 90 may be used on its own in treatment of vascular bifurcations, without a stent. The balloon is shown in the figure in its fully-inflated configuration; during delivery of the balloon through the vascular system, the balloon is typically deflated.

Balloon 90 comprises two parts with different inflation characteristics: an inner part 92, made of semi-compliant material, and collar 94, made of fully-compliant material, which surrounds the proximal end of inner part 92. Typically, balloon 90 comprises a biocompatible polymer material, such as a suitable polyamide. In this embodiment, inner part 92 and collar 94 may be fabricated as separate balloons, with the collar having the general form of a toroid fitted around the inner part. The inner part and collar may share a common inflation port, or they may alternatively have separate inflation ports, enabling the two parts to be inflated to different pressures. Although inner part 92 and collar 94 are seen in FIG. 13 to share a common axis, in an alternative embodiment (not shown in the figures), the axis of collar 94 may be angled relative to the axis of inner part 92. This angled configuration is useful, for example, in treating Y-shaped bifurcations.

Reference is now made to FIGS. 14 and 15, which are schematic side views of the area of a vascular bifurcation, showing the stages in implantation of stent 80 in side vessel 26 using balloon 90, in accordance with an embodiment of the present invention. This implantation procedure may be used in substantially any bifurcation, but it is especially useful for treating the ostia at bifurcations from large arteries, such as the bifurcation of the coronary arteries from the ascending aorta. As shown in FIG. 14, stent 80, in its contracted state, is fitted and crimped over deflated balloon 90, so that the proximal end of the stent extends over inner part 92 of the balloon and over the distal end of collar 94. The stent is delivered by a catheter 96 within a guiding catheter 97 over a guide wire 98, which has been threaded from main vessel 24 into side vessel 26. In this embodiment, balloon 90 has a central lumen with a distal opening for accommodating the guide wire. The central lumen may also permit blood flow in side vessel 26, via the lumen, even when the balloon is fully inflated.

Once stent 80 is in place in side vessel 26, the highly-compliant collar 94 of balloon 90 may be partially inflated, causing the collar to expand to a diameter greater than the diameter of side vessel 26. The operating physician at this stage may push catheter 96 forward, in the distal direction, so that collar 94 engages the ostium. This engagement ensures that stent 80 is properly positioned for expansion. Alternatively, balloon 90 may be designed and operated so that collar 94 is inflated only after inner part 92 and stent 80 have been deployed and expanded in side vessel 26.

Finally, as shown in FIG. 15, balloon 90 is fully inflated, causing the balloon to assume the shape shown in FIG. 13. Inner part 92 pushes distal section 82 of stent 80 outward, to widen vessel 26. Meanwhile, collar 94 spreads struts 84 against the ostium to support the ostium and help to anchor the stent in place. Balloon 90 is then deflated and withdrawn from the body through vessel 24 by using catheter 96.

FIG. 16 is a schematic side view showing construction of a balloon 100, in accordance with an alternative embodiment of the present invention. Balloon 100 comprises a semi-compliant distal part 102 and a fully-compliant proximal part 104. Distal part 102, which has the form of a sleeve, is fitted over proximal part 104 and is then fixed in place, by gluing or fusing with heat or ultrasonic energy, for example. Upon inflation of balloon 100, the proximal end of proximal part 104 (at the upper right in FIG. 16) will inflate to a greater diameter than the distal end, which is constrained by distal part 102. As a result, balloon 100 will assume the shape shown in FIG. 13. In other words, distal part 102 defines the inner part of the balloon, while proximal part 104 defines the collar. Balloon 100 may thus be used in place of balloon 90 in the procedures described above and hereinbelow. Other balloon designs with differential compliance may similarly be used for this purpose and are considered to be within the scope of the present invention.

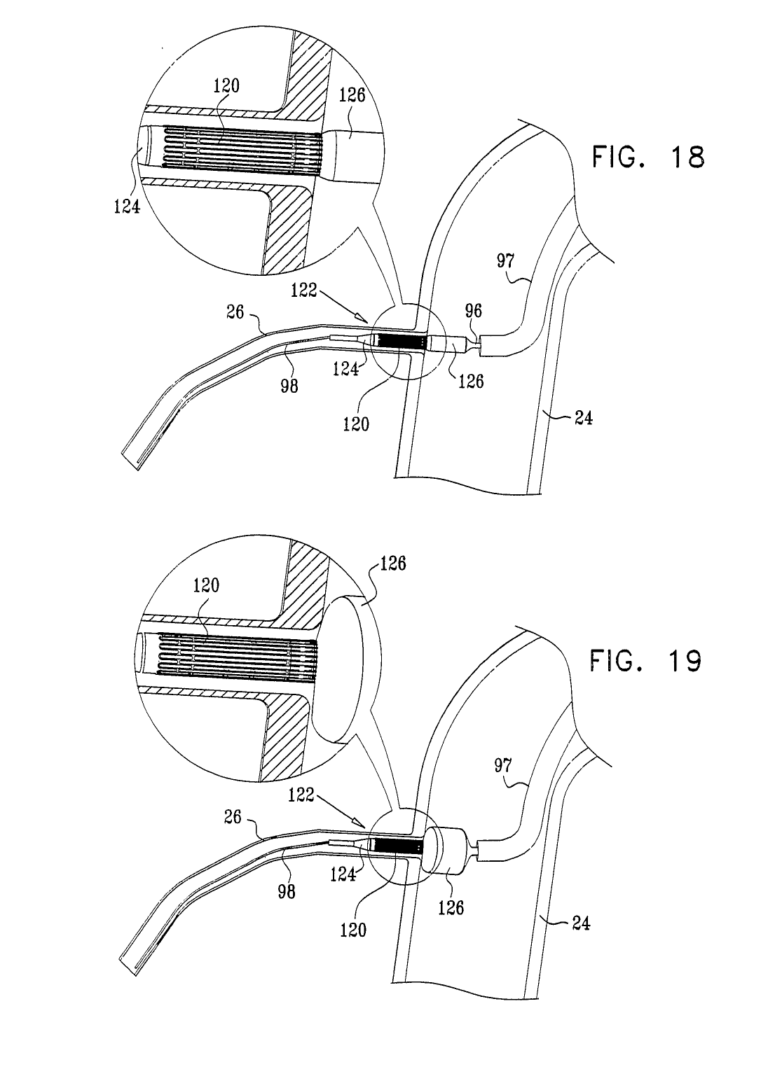

FIG. 17 is a schematic, pictorial illustration of a stent 110, in accordance with an alternative embodiment of the present invention. Stent 110 may be used in place of stent 80 in the procedure described above. As in other embodiments, stent 110 comprises a suitable biocompatible material, which may be capable of eluting a therapeutic substance following implantation.

Stent 110 comprises a distal section 112 and a proximal end 114. The proximal end has a larger number of struts along its perimeter than does the distal section. As a result, proximal end 114 is capable of expanding to a larger diameter than section 112, as illustrated in FIG. 17 (which shows the stent in a partly-expanded configuration). As in the preceding embodiments, stent 110 is maintained in a contracted configuration during delivery of the stent through the vascular system, with an approximately constant diameter over the entire length of the stent, and is then expanded fully in the bifurcation. The stent designs illustrated in FIGS. 12 and 17 are shown only by way of example, and alternative stent designs that permit increased expansion of the proximal end of the stent in the ostial area will be apparent to those skilled in the art. The proximal part may be manufactured as an integral part of the stent in a single process, or it may alternatively be produced separately and attached to the distal section by welding or any other suitable method.

FIGS. 18 and 19 are schematic side views of the area of a vascular bifurcation, showing the stages in implantation of a stent 120 in side vessel 26 using a balloon 122, in accordance with an embodiment of the present invention. Balloon 122 is similar in design to balloon 90, as shown and described above. In this case, however, as shown in FIG. 18, stent 120, in its contracted state, is fitted and crimped over deflated balloon 122 so that the stent extends only over an inner part 124 of the balloon, without extending over a collar 126 of the balloon as in the preceding embodiment. Stent 120 is delivered by catheter 96 over guide wire 98 within guiding catheter 97 into side vessel 26, as shown in FIG. 18.

Proper alignment of a stent in the ostial area of a bifurcation using methods known in the art is a difficult task, typically requiring the use of radiopaque markers to permit the operating physician to visualize the position of the stent. Balloon 122, however, enables the physician to precisely align stent 120 in the ostial area without the need for such markers. For this purpose, the physician inflates collar 126, as shown in FIG. 19. The physician then pushes catheter 96 in the distal direction so as to advance inner part 124 (with stent 120 crimped over it) into side vessel 26. The inflated collar serves as a mechanical stop, halting the distal advance of the stent at its desired location, just inside side vessel 26. The physician then completes the inflation of inner part 124 in order to expand stent 120 in place in the side vessel.

Like balloon 90, balloon 122 has a central lumen with a distal opening for accommodating guide wire 98. The central lumen may also permit blood flow in side vessel 26, via the lumen, even when the balloon is fully inflated.

Various methods may be used to produce the balloons described above:
a) Injection molding using a bifurcated mold with an open or closed protrusion tip (depending upon whether the distal tip of the balloon is to have an opening, typically to accommodate a guide wire, as described above). Balloon raw material, in a liquid, powder or other form, is pre-heated and injected into the mold. After the material has settled inside the mold it is cooled and assumes the appropriate final shape.
b) Blow molding using a bifurcated mold. Balloon raw material in the form of a tube is placed inside the mold (either heated or at room temperature). This tube is then inflated using air, water or other material in order to apply internal pressure that shapes the tube to the geometry of the mold.
c) Blow molding using a bifurcated mold and a vacuum nozzle. This method is similar to that described in the preceding paragraph, with the addition of applying suction through the vacuum nozzle to pull the tube material into the form of the required protrusion, as defined by the shape of the blow mold.
d) Blow molding using a bifurcated mold with a movable inner angled pin (in addition to or instead of applying suction) to direct the tube material in the mold so as to form the required protrusion.
e) Blow molding using a bifurcated telescopic mold. The part of the mold that is used to form the radial protrusion of the balloon is capable of stretching and contracting to form a protrusion of the type shown in FIG. 9, for example.
f) Dipping, using a liquid polymer and a bifurcated balloon model. To form the balloon shown in FIG. 8, for example, one branch of the model (which is used to form the radial protrusion) is capable of being retracted into the model so that the model assumes a cylindrical shape. The balloon model, with the retractable branch extended, is dipped in a tub containing a liquid polymer, which attaches to the surface. The coated model is then removed from the tub and left to dry. After the polymer has hardened and stabilized, the retractable branch is retracted into the model in order to enable removal of the balloon.
g) Blow molding to create the main chamber of the balloon, followed by local treatment at the desired location under appropriate pressure conditions to create the side protrusion. The local treatment may comprise heating, ultrasonic irradiation, or chemical treatment, for example.
   Other methods of manufacture will be apparent to those skilled in the art.

Although the figures in this patent application illustrate certain particular configurations of vessel bifurcations, stents and balloons, these configurations are shown only by way of example. Alternative configurations based on the principles of the present invention will be apparent to those skilled in the art. For example, in some of the embodiments shown in the figures, the vessel bifurcation (and consequently the balloon) has a “T” shape, while in other embodiments the bifurcation and balloon are “Y” shaped. It will be appreciated that each of these embodiments may be adapted for use in bifurcations of both “T” and “Y” types, as well as for other, more complex shapes, according to the configuration of the blood vessels in question. Whereas some of the balloons shown in the figures are configured for insertion over a guide wire, the balloons (and the catheters used to insert them) may alternatively be configured for operation without a guide wire, or for insertion using two or more guide wires if appropriate. The radial protrusion or proximal part of the balloons may also be fabricated as a separate chamber from the main part, as mentioned above, and may thus be inflated via a different channel and to a different pressure, if desired, from the main part of the balloon.

It thus will be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. Apparatus for treatment of a vascular bifurcation, where a first blood vessel meets a second blood vessel, the apparatus comprising a balloon for deployment at the vascular bifurcation, the balloon comprising:
a first part, which has a first inflation characteristic and is adapted to be deployed in the first blood vessel; and
a second part, which has a second inflation characteristic, different from the first inflation characteristic, and is adapted to be deployed in the second blood vessel.

2. The apparatus according to claim 1, wherein the second part is adapted to protrude radially from the first part when the balloon is inflated.

3. The apparatus according to claim 2, wherein the second part is adapted, upon partial inflation of the balloon, to extend into the second blood vessel so as to facilitate alignment of the balloon with the vascular bifurcation.

4. The apparatus according to claim 2, wherein the second part comprises a fan-fold, which is adapted to unfold upon inflation of the balloon so that the second part extends into the second blood vessel.

5. The apparatus according to claim 2, wherein while the balloon is deflated, at least a portion of the second part is contained inside the first part, and wherein the second part is adapted to extend outward from the first part upon inflation of the balloon.

6. The apparatus according to claim 2, and comprising a retraction mechanism, which is coupled to the second part so as to retract the second part radially toward the first part upon deflation of the balloon.

7. The apparatus according to claim 2, and comprising a radiopaque marker in at least a portion of the second part, wherein the marker is configured so as to permit visualization of an alignment of the balloon relative to the bifurcation under angiographic imaging.

8. The apparatus according to claim 7, wherein the radiopaque marker comprises a ring encircling the second part.

9. The apparatus according to claim 2, and comprising a stent, which is fitted over the first part of the balloon and is adapted to be deployed within the first blood vessel by inflation of the balloon, wherein the stent has a radial opening to permit access between the first and second blood vessels, and wherein the second part of the balloon is adapted to protrude radially through the radial opening in the stent.

10. The apparatus according to claim 9, wherein the stent is adapted to elute a therapeutic substance.

11. The apparatus according to claim 1, wherein the inflation characteristic comprises a degree of compliance, such that the first and second parts of the balloon have different, respective degrees of compliance.

12. The apparatus according to claim 11, wherein the second part of the balloon comprises a sleeve, which is secured over a portion of the first part of the balloon so as to constrain the compliance of the portion of the first part.

13. The apparatus according to claim 1, wherein the first and second blood vessels have characteristic first and second diameters, wherein the first diameter is greater than the second diameter, and wherein the first part of the balloon is adapted, upon inflation of the balloon while the second part is deployed in the second blood vessel, to assume an expanded diameter greater than the second diameter.

14. The apparatus according to claim 13, wherein the first part of the balloon is configured as a collar around the second part of the balloon when the balloon is inflated.

15. The apparatus according to claim 14, wherein the first part of the balloon comprises a toroid, which surrounds a portion of the second part of the balloon.

16. The apparatus according to claim 13, wherein the first part of the balloon is adapted, upon the inflation of the balloon, to engage an ostium.

17. The apparatus according to claim 16, and comprising a stent, which is fitted over the second part of the balloon and is adapted to be deployed within the second blood vessel by the inflation of the balloon, the stent comprising a proximal end that is adapted to be expanded to a size greater than the second diameter, and wherein the first part of the balloon is adapted to expand the proximal end of the stent so as to anchor the proximal end against the ostium.

18. The apparatus according to claim 17, wherein the proximal end of the stent comprises a plurality of struts, which are configured to be expanded to the size greater than the second diameter.

19. The apparatus according to claim 17, wherein the stent is adapted to elute a therapeutic substance.

20. The apparatus according to claim 16, and comprising a stent, which is fitted over the second part of the balloon and is adapted to be deployed within the second blood vessel by the inflation of the second part of the balloon, and wherein the first part of the balloon, when expanded, is adapted to serve as a stop against the ostium so as to aid in alignment of the stent within the second blood vessel.

21. The apparatus according to claim 20, wherein the stent is adapted to elute a therapeutic substance.

22. The apparatus according to claim 1, wherein at least one of the first and second parts of the balloon has a lumen passing therethrough to accommodate a guide wire used in the deployment of the balloon.

23. The apparatus according to claim 22, wherein no more than one of the first and second parts of the balloon has the lumen passing therethrough.

24. The apparatus according to claim 1, wherein the first and second parts of the balloon share a common inflation port.

25. The apparatus according to claim 1, wherein the first and second parts of the balloon have separate, respective inflation ports.

26. A method for treatment of a vascular bifurcation, where a first blood vessel meets a second blood vessel, the method comprising:
providing a balloon comprising a first part, which has a first inflation characteristic, and a second part, which has a second inflation characteristic, different from the first inflation characteristic;
deploying the balloon at the vascular bifurcation, such that the first part is deployed in the first blood vessel and the second part is deployed in the second blood vessel; and
inflating the first and second parts of the balloon within the first and second blood vessels, respectively.

27. The method according to claim 26, wherein the second part is adapted to protrude radially from the first part when the balloon is inflated.

28. The method according to claim 27, wherein deploying the balloon comprises partially inflating the balloon so that the second part protrudes radially away from the first part, and aligning the partially-inflated balloon using the second part before completely inflating the balloon.

29. The method according to claim 27, wherein the second part comprises a fan-fold, and wherein inflating the first and second parts comprises unfolding the fan-fold so that the second part extends into the second blood vessel.

30. The method according to claim 27, wherein while the balloon is deflated, at least a portion of the second part is contained inside the first part, and wherein inflating the first and second parts causes the second part to extend outward from the first part.

31. The method according to claim 27, and comprising retracting the second part radially toward the first part upon deflation of the balloon.

32. The method according to claim 27, wherein a radiopaque marker is positioned in at least a portion of the second part, and wherein deploying the balloon comprises using the marker under angiographic imaging so as to visualize of an alignment of the balloon relative to the bifurcation.

33. The method according to claim 32, wherein the radiopaque marker comprises a ring encircling the second part.

34. The method according to claim 27, and comprising fitting a stent over the first part of the balloon, wherein the stent has a radial opening to permit access between the first and second blood vessels, wherein inflating the first and second parts of the balloon comprises deploying the stent within the first blood vessel by inflation of the balloon, which causes the second part of the balloon to protrude radially into the second blood vessel through the radial opening in the stent.

35. The method according to claim 34, wherein the stent is adapted to elute a therapeutic substance.

36. The method according to claim 26, wherein the inflation characteristic comprises a degree of compliance, such that the first and second parts of the balloon have different, respective degrees of compliance.

37. The method according to claim 36, wherein the second part of the balloon comprises a sleeve, which is secured over a portion of the first part of the balloon so as to constrain the compliance of the portion of the first part.

38. The method according to claim 26, wherein the first and second blood vessels have characteristic first and second diameters, wherein the first diameter is greater than the second diameter, and wherein inflating the first and second parts of the balloon comprises inflating the first part to an expanded diameter greater than the second diameter.

39. The method according to claim 38, wherein the first part of the balloon is configured as a collar around the second part of the balloon when the first part of the balloon is inflated.

40. The method according to claim 39, wherein the first part of the balloon comprises a toroid, which surrounds a portion of the second part of the balloon.

41. The method according to claim 38, wherein deploying the balloon comprises positioning the balloon so that upon inflation of the balloon, the first part of the balloon engages an ostium.

42. The method according to claim 41, and comprising fitting a stent over the second part of the balloon, wherein inflating the first and second parts of the balloon comprises deploying the stent within the second blood vessel by the inflation of the second part of the balloon, while expanding a proximal end of the stent to a size greater than the second diameter by the inflation of the first part of the balloon so as to anchor the proximal end against the ostium.

43. The method according to claim 42, wherein the proximal end of the stent comprises a plurality of struts, and wherein expanding the proximal end comprises spreading the struts.

44. The method according to claim 42, wherein the stent is adapted to elute a therapeutic substance.

45. The method according to claim 41, and comprising fitting a stent over the second part of the balloon, wherein positioning the balloon comprises:
aligning the stent on the second part of the balloon inside the second blood vessel after inflating the first part of the balloon to the expanded diameter, so that the first part of the balloon serves as a stop against the ostium; and
after aligning the second part of the balloon, expanding the second part of the balloon so as to deploy the stent within the second blood vessel.

46. The method according to claim 45, wherein the stent is adapted to elute a therapeutic substance.

47. The method according to claim 26, wherein deploying the balloon comprises passing a guide wire through a lumen in at least one of the first and second parts of the balloon, and deploying the balloon at the vascular bifurcation over the guide wire.

48. The method according to claim 47, wherein no more than one of the first and second parts of the balloon has the lumen passing therethrough.

49. The method according to claim 26, wherein inflating the first and second parts of the balloon comprises inflating both of the parts of the balloon through a common inflation port.

50. The method according to claim 26, wherein inflating the first and second parts of the balloon comprises inflating the first and second parts of the balloon through separate, respective inflation ports.

51. Apparatus for treatment of a vascular bifurcation, where a first blood vessel meets a second blood vessel, the apparatus comprising:
a balloon for deployment at the vascular bifurcation, the balloon comprising a first part, which has a first inflation characteristic and is adapted to be deployed in the first blood vessel, and a second part, which has a second inflation characteristic, different from the first inflation characteristic, and is adapted to be deployed in the second blood vessel; and
a catheter, having a distal end to which the balloon is coupled, and which is adapted to pass through the first blood vessel so as to deploy the balloon at the bifurcation.

52. A method for manufacturing an intravascular balloon, comprising:
fabricating a first part of the balloon so as to have a first inflation characteristic; and
fabricating a second part of the balloon, coupled to the first part, so as to have a second inflation characteristic, which is different from the first inflation characteristic.

53. The method according to claim 52, wherein fabricating the second part of the balloon comprises fabricating the second part as a bifurcation from the first part.

54. The method according to claim 52, wherein fabricating the second part of the balloon so that the first part forms a collar around the second part when the balloon is inflated.

55. The method according to claim 52, wherein the first and second inflation characteristics respectively comprise different, first and second degrees of compliance.

56. The method according to claim 55, wherein fabricating the second part of the balloon comprises securing a sleeve over a portion of the first part of the balloon so as to constrain the compliance of the portion of the first part.

57. The method according to claim 52, and comprising fitting a stent over at least one of the first and second parts of the balloon.

58. The method according to claim 52, wherein fabricating the first and second parts of the balloon comprises fabricating at least one of the first and second parts by injection molding using a bifurcated mold.

59. The method according to claim 52, wherein fabricating the first and second parts of the balloon comprises fabricating at least one of the first and second parts by blow molding using a bifurcated mold.

60. The method according to claim 59, wherein the bifurcated mold comprises a telescopic mold.

61. The method according to claim 59, wherein fabricating the at least one of the first and second parts comprises applying at least one of suction and an angled pin to direct material into the bifurcated mold.

62. The method according to claim 52, wherein fabricating the first and second parts of the balloon comprises fabricating the first and second parts by dipping a bifurcated model in a liquid polymer.

63. The method according to claim 52, wherein fabricating the first and second parts of the balloon comprises fabricating the first part of the balloon, and then applying a local treatment to an area of the first part in order to form the second part.

64. A vascular stent, comprising:
a distal section, which is adapted to be deployed and expanded within a blood vessel of a given diameter in a location adjacent to an ostium; and
a proximal section, which is adapted to be expanded against the ostium to a size greater than the given diameter so as to anchor the proximal section against the ostium.

65. The stent according to claim 64, wherein the distal section of the stent comprises a first number of struts along a perimeter of the stent, and wherein the proximal section of the stent comprises a second number of the struts, greater than the first number.

66. The stent according to claim 64, wherein the proximal section comprises a plurality of struts, which are configured to bend outward so as to engage the ostium.

67. The stent according to claim 64, wherein at least one of the distal and proximal sections is adapted to elute a therapeutic substance.

68. Apparatus for treatment of a vascular bifurcation, where a first blood vessel meets a second blood vessel, the apparatus comprising a balloon for deployment at the vascular bifurcation, the balloon comprising:
a first part, which is adapted to be deployed in the first blood vessel; and
a second part, which is adapted to protrude radially from the first part when the balloon is inflated so as to facilitate alignment of the balloon with the vascular bifurcation.

69. The apparatus according to claim 68, and comprising a stent, which is fitted over the first part of the balloon and is adapted to be deployed within the first blood vessel by inflation of the balloon, wherein the stent has a radial opening to permit access between the first and second blood vessels, and wherein the second part of the balloon is adapted to protrude radially through the radial opening in the stent.

70. The apparatus according to claim 69, wherein the stent comprises struts over the radial opening, and wherein the second part of the balloon is adapted to open the struts outward when the balloon is inflated.

71. The apparatus according to claim 68, and comprising a radiopaque marker in at least a portion of the second part, wherein the marker is configured so as to permit visualization of an alignment of the balloon relative to the bifurcation under angiographic imaging.

72. Apparatus for treatment of a vascular bifurcation, where a first blood vessel meets a second blood vessel, wherein the first and second blood vessels have characteristic first and second diameters, wherein the first diameter is greater than the second diameter, the apparatus comprising a balloon for deployment at the vascular bifurcation, the balloon comprising:
an inner part, which is adapted to be deployed in the second blood vessel; and
a collar around the inner part, which is adapted, upon inflation of the balloon while the second part is deployed in the second blood vessel, to assume an expanded diameter greater than the second diameter.

73. The apparatus according to claim 72, wherein the collar comprises a toroid, which surrounds a portion of the inner part of the balloon.

74. The apparatus according to claim 72 or 73, wherein the collar is adapted, upon the inflation of the balloon, to engage an ostium.

75. The apparatus according to claim 74, and comprising a stent, which is fitted over the inner part of the balloon and is adapted to be deployed within the second blood vessel by the inflation of the balloon, the stent comprising a proximal end that is adapted to be expanded to a size greater than the second diameter, and wherein the collar is adapted to expand the proximal end of the stent so as to anchor the proximal end against the ostium.

76. The apparatus according to claim 74, and comprising a stent, which is fitted over the inner part of the balloon and is adapted to be deployed within the second blood vessel by the inflation of the second part of the balloon, and wherein the collar, when expanded, is adapted to serve as a stop against the ostium so as to aid in alignment of the stent within the second blood vessel.

77. A method for treatment of a vascular bifurcation, where a first blood vessel meets a second blood vessel, the method comprising:
providing a balloon comprising a first part, which has a first inflation characteristic, and a second part, which is adapted to protrude radially from the first part when the balloon is inflated;
deploying the balloon in a vicinity of the vascular bifurcation, such that the first part is deployed in the first blood vessel;
partially inflating the balloon in the vicinity of the vascular bifurcation so that the second part protrudes radially away from the first part;
aligning the second part of the partially-inflated balloon with the second blood vessel; and
fully inflating the balloon after aligning the second part.

78. The method according to claim 77, and comprising fitting a stent over the first part of the balloon, wherein the stent has a radial opening to permit access between the first and second blood vessels, wherein fully inflating the balloon comprises deploying the stent within the first blood vessel by inflation of the balloon, which causes the second part of the balloon to protrude radially into the second blood vessel through the radial opening in the stent.

79. The method according claim 78, wherein the stent comprises struts over the radial opening, and wherein fully inflating the balloon causes the second part of the balloon to open the radial struts outward against an ostium.

80. A method for treatment of a vascular bifurcation, where a first blood vessel meets a second blood vessel, wherein the first and second blood vessels have characteristic first and second diameters, wherein the first diameter is greater than the second diameter, the method comprising:
providing a balloon comprising an inner part and a collar around the inner part;
deploying the balloon at the vascular bifurcation, such that the inner part is deployed in the first blood vessel and the collar is deployed in the second blood vessel; and
inflating the balloon so that the collar expands to an expanded diameter greater than the second diameter and engages an ostium.

81. The method according to claim 80, and comprising fitting a stent over the inner part of the balloon, wherein deploying the balloon comprises deploying the stent within the second blood vessel, and wherein inflating the balloon causes the collar to expand a proximal end of the stent to a size greater than the second diameter so as to anchor the proximal end against the ostium.

82. The method according to claim 80, and comprising fitting a stent over the inner part of the balloon, wherein deploying the balloon comprises:
aligning the stent on the inner part of the balloon inside the second blood vessel after inflating the collar to the expanded diameter, so that the collar serves as a stop against the ostium; and
after aligning the inner part of the balloon, expanding the second part of the balloon so as to deploy the stent within the second blood vessel.
